(19) United States
(12) Patent Application Publication
(10) Pub. No.: US 2009/0156629 A1
(43) Pub. Date: Jun. 18, 2009

(54) INDENOISOQUINOLINE-RELEASABLE POLYMER CONJUGATES

(75) Inventors: Hong Zhao, Edison, NJ (US); Ying Gao, Pennington, NJ (US)

Correspondence Address: LUCAS & MERCANTI, LLP, 475 PARK AVENUE SOUTH, 15TH FLOOR, NEW YORK, NY, 10016, US

(73) Assignee: Enzon Pharmaceuticals, Inc., Bridgewater, NJ (US)

(21) Appl. No.: 12/301,346

(22) PCT Filed: Jun. 8, 2007
(86) PCT No.: PCT/US07/70801

§ 371 Date: Dec. 15, 2008

Related U.S. Application Data
(60) Provisional application No. 60/804,388, filed on Jun. 9, 2006.

(51) Int. Cl. A61K 47/48; C08G 65/333

(52) U.S. Cl. 514/283; 525/408

(57) ABSTRACT

The present invention provides releasably-linked indenoisoquinoline polymer conjugates. Methods of making the conjugates and methods of treating mammals using the same are also disclosed.

### CROSS-REFERENCE TO RELATED APPLICATION

This application claims the benefit of priority from U.S. Provisional Patent Application Ser. No. 60/804,388 filed Jun. 9, 2006, the contents of which are incorporated herein by reference.

### BACKGROUND OF THE INVENTION

Camptothecin and camptothecin analogs are known as topoisomerase I inhibitors. They have shown to have anti-tumor efficiencies. Despite the anti-tumor efficiencies, camptothecin and its analogs have some disadvantageous properties. The presence of the lactone ring within the structure has limited their clinical utility. See below.

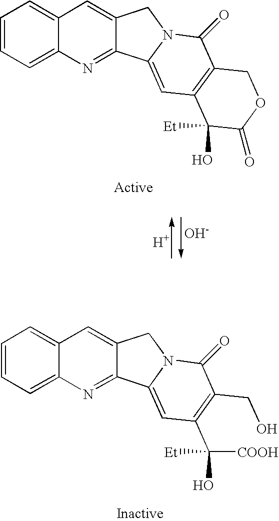

Over the years, there have been significant attempts to provide topoisomerase I inhibitors having potential anti-tumor efficiencies and chemical stability. Some attempts have led to the discovery of indenoisoquinolines which have shown good in vitro anti-tumor efficacy. Indenoisoquinolines do not have lactone rings and show significant chemical stability. Despite the stability and hi vitro anti-tumor activity, the clinical utility of indenoisoquinolines has been severely limited due to its poor water solubility. Indenoisoquinolines are described, for example, in US Patent Application Publication No. 2006/0025595.

In view of the forgoing drawbacks, there continues to be a need to provide topoisomerase I inhibitors having desirable therapeutic activity and water solubility properties. It would also be desirable to provide topoisomerase I inhibitors which are substantially non-antigenic. It would also be desirable to provide topoisomerase I inhibitors having sufficient bioavailability without being prematurely eliminated from the body through the kidney or reticular enthothelial system, etc. It would also be desirable to provide topoisomerase inhibitors having controllable circulation half-lives. The present invention addresses these need and other needs.

### SUMMARY OF THE INVENTION

hi one aspect of the invention, there are provided releasably-linked indenoisoquinoline polymer conjugates. The releasably-linked indenoisoquinoline polymer conjugates have the structure of Formula (I):

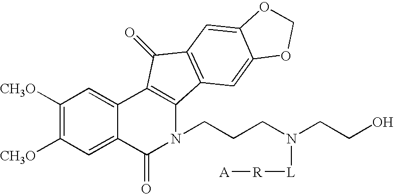

wherein

A is a capping group such as a methyl group or

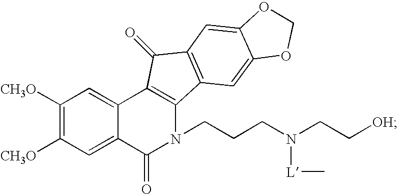

R is a substantially non-antigenic water-soluble polymer such as a polyethylene glycol; and

L and L′ are independently selected releasable linkers.

Some particularly preferred conjugates are of the structure:

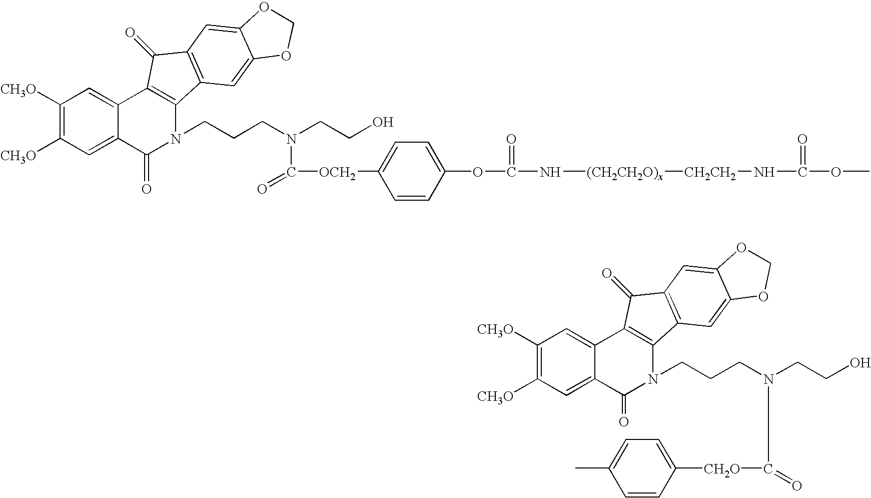

wherein (x) is an integer from about 10 to about 2300. (x) is a positive integer selected so that the PEG preferably has a molecular weight of preferably greater than 10,000. In alternative aspects of the invention, the molecular weight of tire PEG is about 20,000 or 40,000.

Further aspects of the invention include compounds of tire formula shown below:

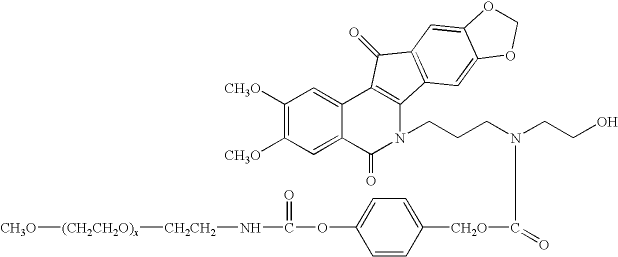

wherein x has the same definition as set forth above.

In still further aspects of the invention, there are provided pharmaceutically acceptable salts of the foregoing as well as pharmaceutically-acceptable formulations containing the same. Methods of treatment are also contemplated wherein a therapeutically effective amount of a polymer conjugate as described herein is administered to a patient i.e. a mammal, in need thereof. In yet further aspects, there are provided methods of preparing the releasably-linked indenoisoquinoline polymer conjugates described herein.

One advantage provided by the present invention includes the ability to provide excellent aqueous solubility to a indenoisoquinoline showing favorable clinical properties, 6-[3-(2-hydroxyethyl)amino-1-propyl]-5,6-dihydro-2,3-dimethoxy-8,9-methylenedioxy-5,11-dioxo-11H-indeno[1,2-c]isoquinoline), also known as MJ III-65. The free base form of MJ III-65 has the structure:

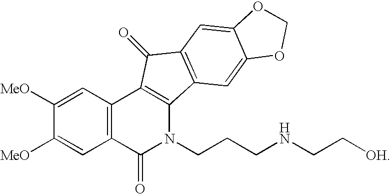

For purposes of the present invention, “IndQ” shall be understood to mean 6-[3-(2-hydroxyethyl)amino-1-propyl]-5,6-dihydro-2,3-dimethoxy-8,9-methylenedioxy-5,11-dioxo-11H-indeno[1,2-c]isoquinoline) and salt forms thereof.

The compounds described herein also provide improved pharmacokinetic profiles and thereby provide in vivo anti-tumor efficiencies. Furthermore, the present invention provides pharmaceutical formulations including the compounds described herein. The formulations facilitate administration into mammals via intravenous administration. Upon administration, the parent compound, IndQ is liberated therefrom. Without being bound by any theories, the following schematically shows controlled release of the IndQ parent drug from the releasably-linked IndQ polymer conjugates.

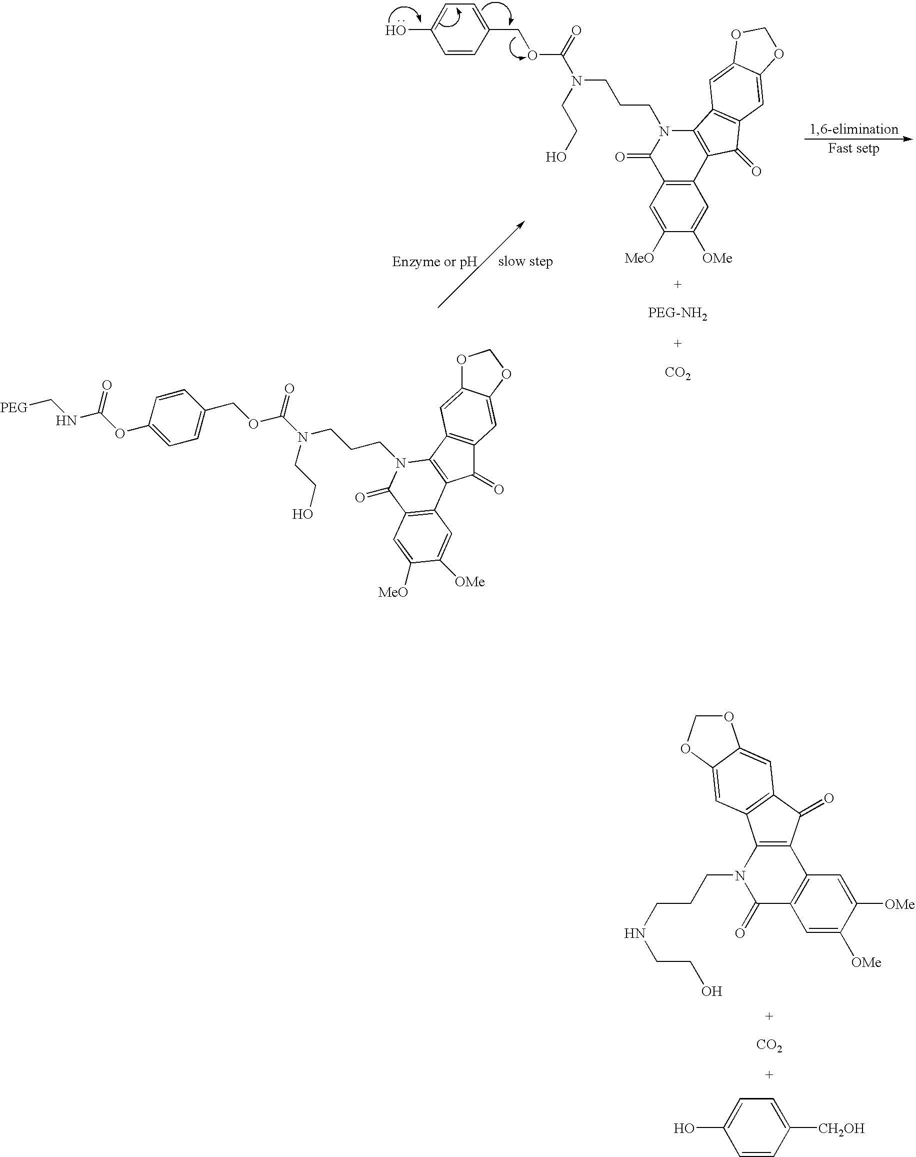

Other and further advantages will be apparent from the following description and examples.

For purposes of the present invention, the term “alkyl” shall be understood to include straight, branched, substituted, e.g. halo-, alkoxy-, nitro-, C₁₋₁₂, but preferably C₁₋₄ alkyls, C₃₋₈ cycloalkyls or substituted cycloalkyls, etc.

For purposes of the present invention, the term “substituted” shall be understood to include adding or replacing one or more atoms contained within a functional group or compound with one or more different atoms.

For purposes of the present invention, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercapto alkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

The terms “effective amounts” and “sufficient amounts” for purposes of the present invention shall mean an amount which achieves a desired effect or therapeutic effect as such effect is understood by those of ordinary skill in the art.

### DETAILED DESCRIPTION OF THE INVENTION

### A. Overview

In one aspect of the invention, the present invention provides compounds of Formula (I):

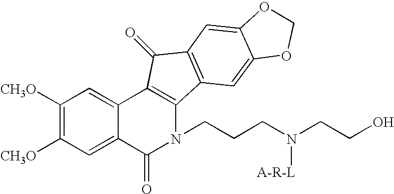

wherein

A is a capping group or

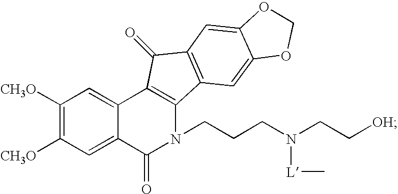

R is a substantially non-antigenic water-soluble polymer; and

L and L′ are independently selected releasable linkers.

In one aspect of the present invention, the compounds described herein include benzyl elimination system-based releasable linkers (hereafter, “RNL” linkers or “RNL-based” linkers). The releasable linkers L and L′ are independently of the Formula (II)

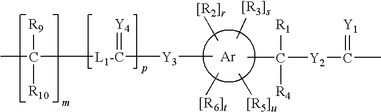

wherein

L₁ is a bifunctional linking moiety;

Y₁₋₄ are independently O, S, or NR₁₂;

R₁, R₄, R₉, R₁₀, and R₁₂, are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;

R₂, R₃, R₅ and R₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;

Ar is an aromatic moiety which when included in Formula (II) forms a multi-substituted aromatic hydrocarbon or a multi-substituted heteroaromatic group;

(r), (s), (t), and (u) are independently zero or one; and

(m) and (p) are independently zero a positive integer.

The compounds of Formula (III) preferably include one particular derivative of indenoisoquinoline, 6-[3-(2-hydroxyethyl)amino-1-propyl]-5,6-dihydro-2,3-dimethoxy-8,9-methylenedioxy-5,11-dioxo-11H-indeno[1,2-c]isoquinoline), known as MJ III-65. A free base of MJ III-65 is identified as NSC 706743 and a HCl salt form as NSC 706744. The free base form of MJ III-65 has the structure:

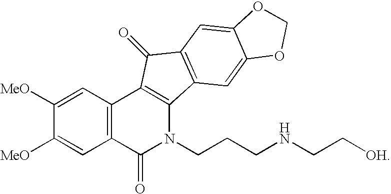

In certain aspects of the invention, there are provided compounds having Formula (III)

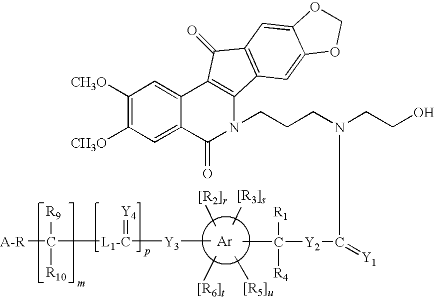

wherein

A is a capping group or

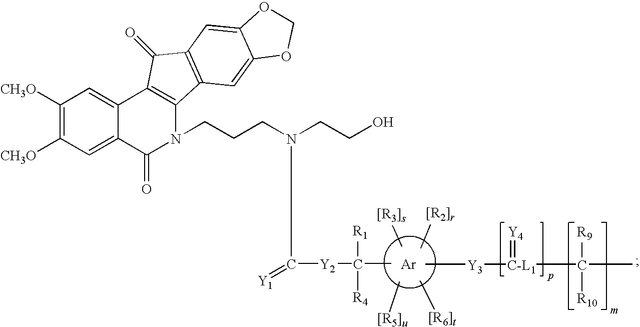

R is a substantially non-antigenic water-soluble polymer;

L₁ is a bifunctional linking moiety;

Y₁₋₄ are independently O, S, or NR₁₂;

R₁, R₄, R₉, R₁₀, and R₁₂, are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;

R₂, R₃, R₅ and R₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;

Ar is an aromatic moiety which when included in Formula (II) forms a multi-substituted aromatic hydrocarbon or a multi-substituted hetero aromatic group;

(r), (s), (t), and (u) are independently zero or one; and

(m) and (p) are independently zero or a positive integer.

In some particular aspects, (m) and (p) are independently 0-6 and preferably 1. In other preferred embodiments, (m) and (p) are zero. In yet further preferred aspects, R is not the PEG derivative corresponding to —C(═Y₂₁)—(CH₂)ₙ—O—(CH₂CH₂O)ₓ—CH₃ when m=0, p=0 and Y₃=NH; nor is R the PEG derivative —CH₃—O—(CH₂CH₂O)ₓ—(CH₂)ₙ— when m=0, p=1, L₁=OCH₂, Y₄═O and Y₃═NH. In each case (n) is zero or a positive integer; Y₂₁ is O, S or NR₁₂; and (x) represents the degree of polymerization described herein.

In some other preferred embodiments, (m) and (p) are 1. In another particular aspect, R₁, R₄, R₉ and R₁₀ are all preferably hydrogen. In yet another particular aspect, Y₁₋₄ are O, and (m) is 0.

In those aspects of the invention where A is a capping group, it will be understood by those of ordinary skill that such groups include moieties such as hydrogen, NH₂, OH, CO₂H, C₁₋₆ alkyls or substituted alkyls, etc, C₁₋₆ alkoxy substituted alkoxys, etc, dialkyl acyl urea alkyls and the like. In some aspects of the invention the capping group is preferably CH₃ or OCH₃. Alternatively, A is

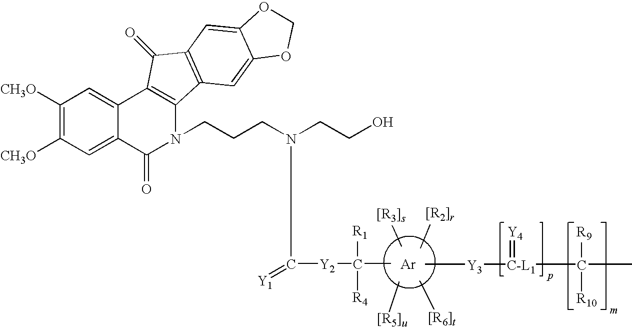

wherein all variables are as previously defined so that the bis derivative is formed.

In other particular aspects, there are provided compounds having the formula:

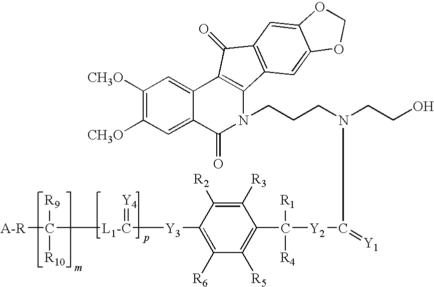

wherein all variables are as previously defined so that the bis derivative is formed. The bis form is sometimes described as the Δform of the polymer conjugates which allow double-loading of the IndQ on the polymer. Within this aspect, some particularly preferred compounds of the invention include those of the formula

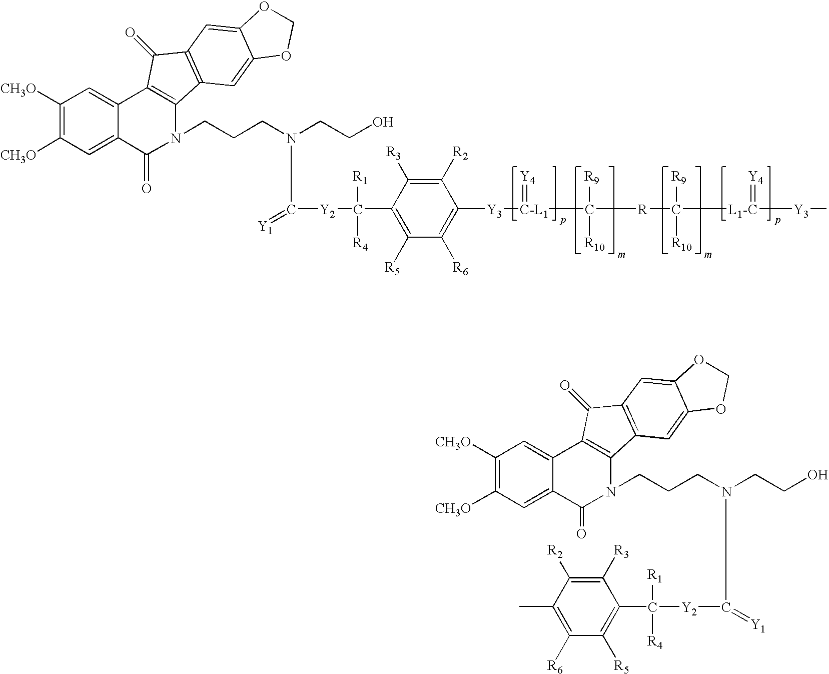

wherein all variables are as previously defined.

In some alternative aspects, there are provided ortho-substituted compounds having the formula:

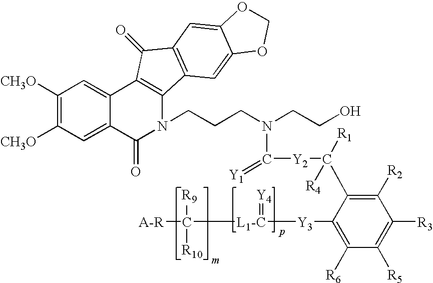

The ortho-substituted compounds further include

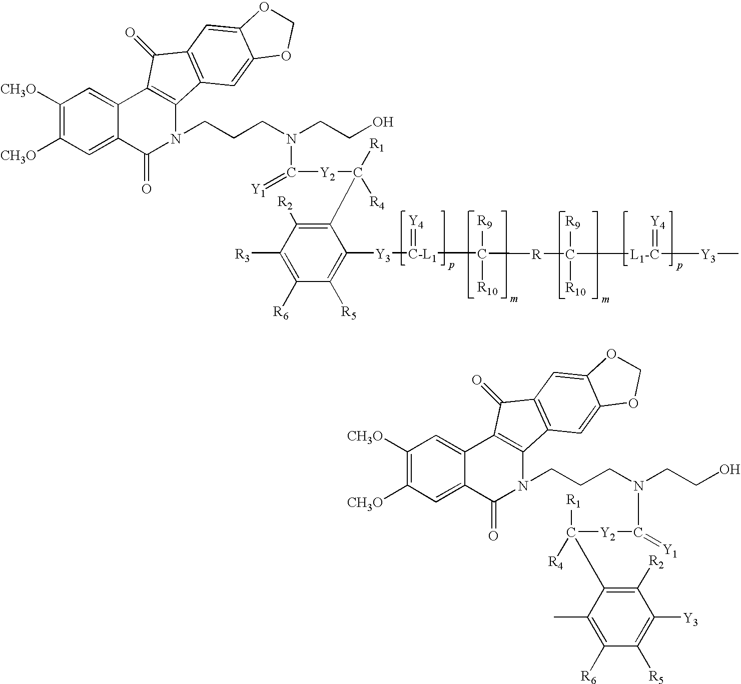

where all variables are as previously defined.

In some aspects of the Invention, the compounds described herein include a linear, terminally branched or multi-armed polyalkylene oxide. In some preferred embodiments of the invention, the polyalkylene oxide includes polyethylene glycol and polypropylene glycol.

In yet more preferred aspects, R can be —C(═Y₂₁)—(CH₂)ₙ—O—(CH₂CH₂O)ₓ-A, or —C(═Y₂₁)—Y₂₂—(CH₂)ₙ—O—(CH₂CH₂O)ₓ-A

wherein

(n) is zero or a positive integer;

Y₂₁₋₂₂ are independently O, S or NR₁₂;

(x) represents the degree of polymerization and

A is as previously defined.

### B. Substantially Non-Antigenice Water-Soluble Polymers

Polymers employed in the compounds described herein are preferably water soluble polymers and substantially non-antigenic such as polyalkylene oxides (PAO's). The polyalkylene oxide has an average molecular weight from about 2,000 to about 100,000 daltons, preferably front about 10,000 to about 80,000 daltons and more preferably from about 20,000 to about 40,000 or 60,000 daltons. In some alternative embodiments, the compounds described herein include the polyalkylene oxide having an average molecular weight of about 40,000 daltons. The polyalkylene oxide includes polyethylene glycols and polypropylene glycols. More preferably, the polyalkylene oxide includes polyethylene glycol, PEG is generally represented by the structure:

—O—(CH₂CH₂O)ₓ—

where (x) represents the degree of polymerization for the polymer, i.e. the number of repeating units in the polymer chain and is dependent on the molecular weight of the polymer. For purposes of illustration and not limitation, the polyethylene glycol (PEG) residue portion of the invention can be selected from among:

—C(—Y₂₁)—(CH₂)ₙ—O—(CH₂CH₂O)ₓ-A, and

—C(═Y₂₁)—Y₂₂—(CH₂)ₙ—O—(CH₂CH₂O)ₓ-A

wherein

(n) is zero or a positive integer, preferably 1-6, and more preferably 1;

Y₂₁₋₂₂ are independently O, S or NR₁₂; and

(x) is an integer from about 10 to about 2,300.

Alternatively, the PEG portion can be:

—X₁₁—(CH₂CH₂O)ₓ—CH₂CH₂X₁₁—,

—X₁₁—(CH₂CH₂O)ₓ—CH₂C(═Y₁₁)—X₁₁—,

—X₁₁—C(═Y₁₁)—(CH₂)_{a′}—Y₁₂—(CH₂CH₂O)ₓ—CH₂CH₂—Y₁₂—(CH₂)_{a′}—C(═Y₁₁)—X₁₁—, and

—X₁₁—(CR₃₁R₃₂)_{a′}—Y₁₂—(CH₂)_{b′}—O—(CH₂CH₂O)ₓ—(CH₂)_{b′}—Y₁₂—(CR₃₁R₃₂)_{a′}—X₁₁—,

wherein:

each X₁₁, is independently absent, O, S, SO, SO₂ or NR₃₃;

each Y₁₁ is O, S, or NR₃₃;

each Y₁₂ is independently absent, O, S, or NR₃₃;

R₃₁₋₃₃ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy,
wherein the substituents are selected from the group consisting of acyl, amino, amido, amidine, araalkyl, aryl, azido, alkylmercapto, arylmercapto, carbonyl, carboxylate, cyano, ester, ether, formyl, halogen, heteroaryl, heterocycloalkyl, hydroxy, imino, nitro, thiocarbonyl, thioester, thioacetate, thioformate, alkoxy, phosphoryl, phosphonate, phosphinate, silyl, sulfhydryl, sulfate, sulfonate, sulfamoyl, sulfonamide, and sulfonyl;

(a′) and (b′) are independently zero or a positive integer, preferably 1-6 and more preferably 1; and

(x) is an integer from about 10 to about 2300.

Branched or U-PEG derivatives are described in U.S. Pat. Nos. 5,643,575, 5,919,455, 6,113,906 and 6,566,506, the disclosure of each incorporated herein by reference. A non-limiting list of such polymers corresponds to polymer systems (v)-(ix) with the following structures:

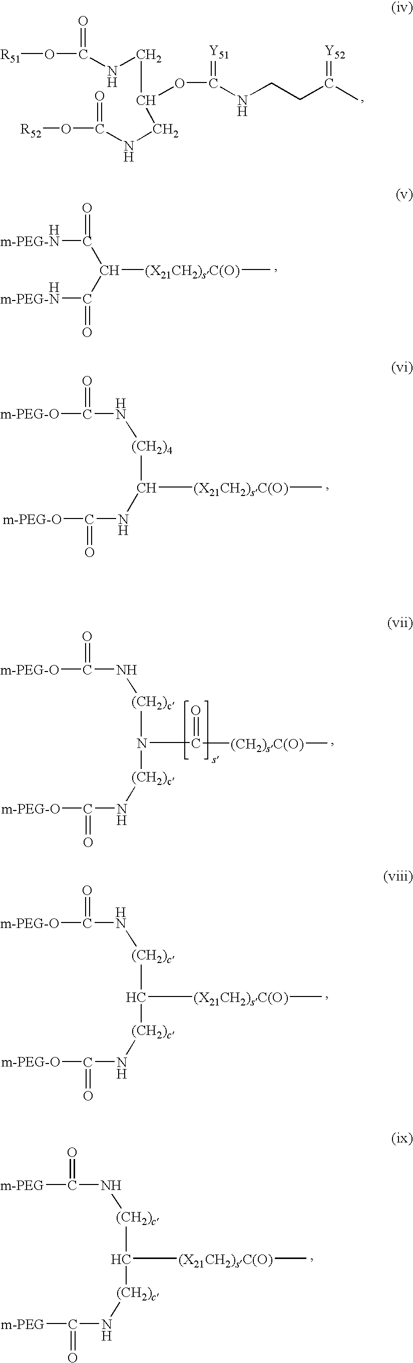

wherein:

R₅₁₋₅₂ are polyalkylene oxide;

Y₅₁₋₅₂ are independently selected from O, S and NR₁₂;

X₂₁ is O, NR₁₂, S, SO or SO₂

(c′) and (t′) are independently 0 or a positive integer;

(s′) is 0 or 1;

mPEG is H₃CO(—CH₂CH₂O)ₓ—
wherein (x) is a positive integer selected so that a total molecular weight of the polymer is from about 2,000 to about 100,000 daltons, and preferably from about 20,000 to about 60,000 daltons. R₁₂ is previously defined.

In yet another aspect, the polymers include multi-arm PEG-OH or “star-PEG” products such as those described in NOF Corp. Drug Delivery System catalog, Ver. 8, April 2006, the disclosure of which is incorporated herein by reference. The polymers with releasable linkers can be converted into a suitably activated polymer, using the activation techniques described in U.S. Pat. No. 5,122,614 or 5,808,096. Specifically, such PEG can be of the formula:

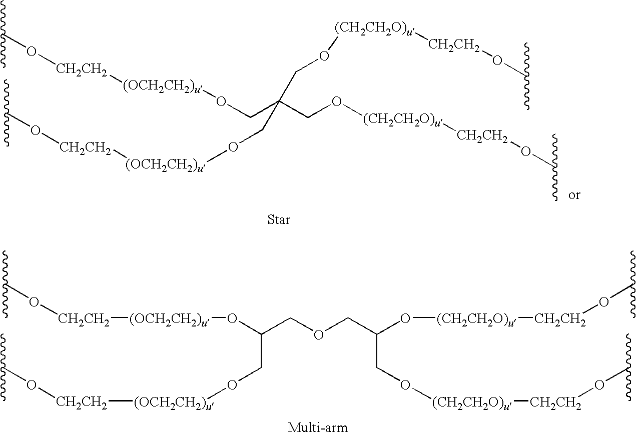

wherein:

(u′) is an integer from about 4 to about 455, to preferably provide polymers having a total molecular weight of from about 20,000 to about 60,000; and up to 3 terminal portions of the residue is/are capped with a methyl or other lower alkyl

In some preferred embodiments, all 4 of the PEG arms are converted to suitable leaving groups, for facilitating attachment to IndQ. Such compounds prior to conversion include:

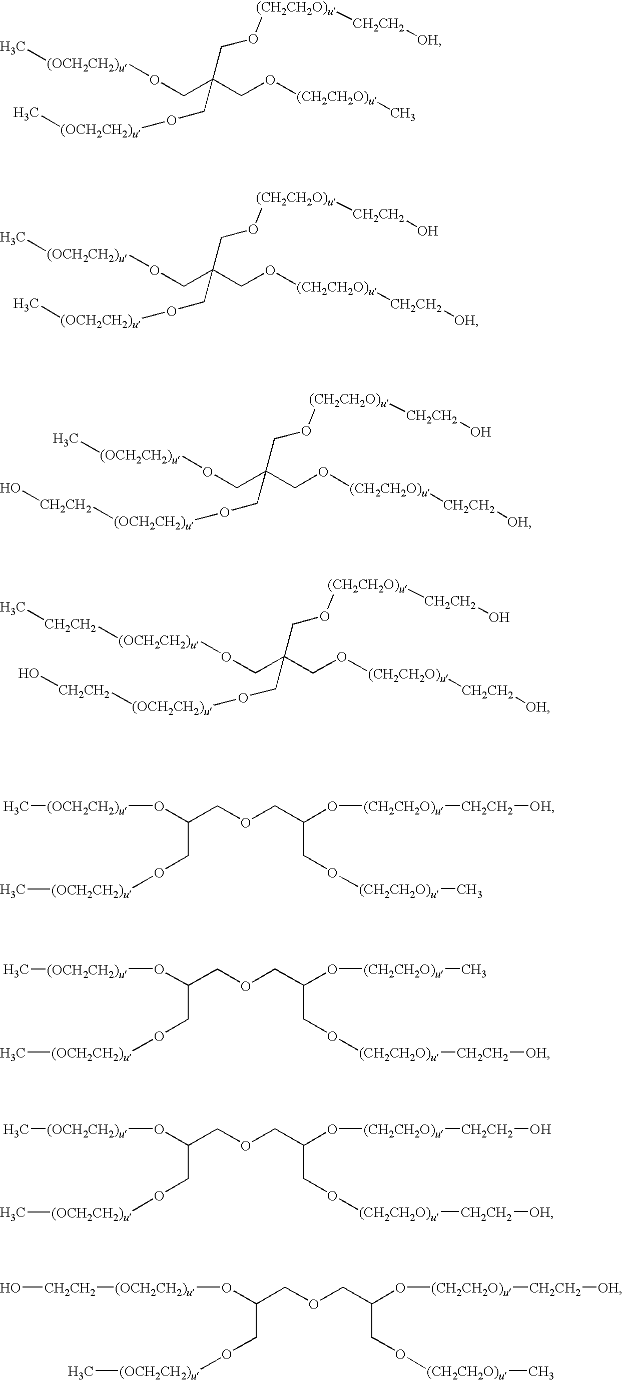

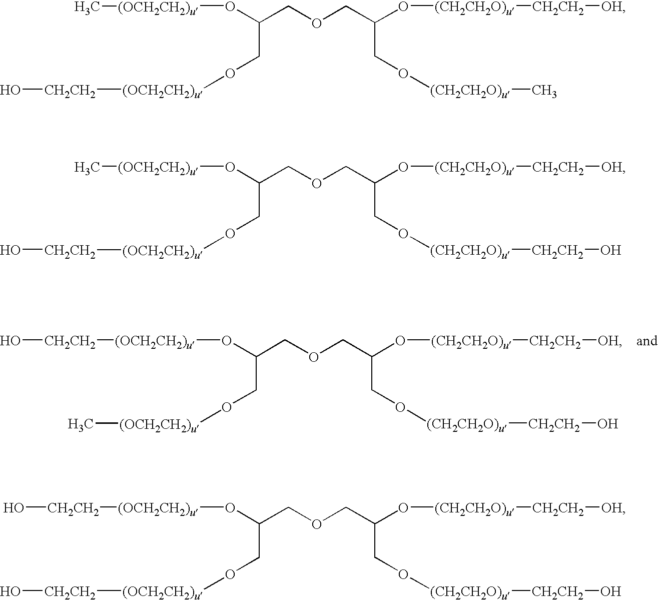

Suitable star or multi-arm polymers will vary substantially by weight. Such polymers having total average molecular weights ranging front about 2,000 to about 100,000 daltons are usually selected for purposes of the present invention. Molecular weights of from about 20,000 to about 60,000 daltons are preferred and 40,000 daltons is particularly preferred.

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In a further embodiment, and as an alternative to PAO-based polymers, one or more effectively non-antigenic materials such as dextran, polyvinyl alcohols, carbohydrate-based polymers, hydroxypropylmethacrylamide (HPMA), polyalkylene oxides, and/or copolymers thereof can be used. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

In some aspects, polymers having terminal amine groups can be employed to make the polymer conjugates. The methods of preparing polymers containing terminal amines in high purity are described in U.S. patent application Ser. Nos. 11/508,507 and 11/537,172, the contents of each of which are incorporated by reference. For example, polymers having azides react with phosphine-based reducing agent such as triphenylphosphine or an alkali metal borohydride reducing agent such as NaBH₄. Alternatively, polymers including leaving groups react with protected amine salts such as potassium salt of methyl-tert-butyl imidodicarbonate (KNMeBoc) or the potassium salt of di-tert-butyl imidodicarbonate (KNBoc₂) followed by deprotecting the protected amine group. The purity of the polymers containing the terminal amines formed by these processes is greater than about 95% and preferably greater than 99%.

In yet alternative aspects, polymers having terminal carboxylic acid groups can be employed in the polymer conjugates. Methods of preparing polymers having terminal carboxylic acids in high purity arc described in U.S. patent application Ser. No. 11/328,662, the contents of which are incorporated herein by reference. The methods include first preparing a tertiary alkyl ester of a polyalkylene oxide followed by conversion to the carboxylic acid derivative thereof. The first step of the preparation of the PAO carboxylic acids of the process includes forming an intermediate such as t-butyl ester of polyalkylene oxide carboxylic acid. This intermediate is formed by reacting a PAO with a t-butyl haloacetate in the presence of a base such as potassium f-butoxide. Once the t-butyl ester intermediate has been formed, the carboxylic acid derivative of the polyalkylene oxide can be readily provided in purities exceeding 92%, preferably exceeding 97%, more preferably exceeding 99% and most preferably exceeding 99.5% purity.

### C. Releasable Linkers

The compounds of the present invention employ releasable linkers. Such releasable linkers are based on benzyl elimination systems. In certain embodiments, L and L′ are independently selected embodiments of Formula (II)

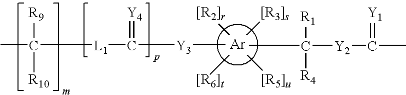

wherein

L₁ is a bifunctional linking moiety;

Y₁₋₄ are independently O, S, or NR₁₂;

R₁, R₄, R₉, R₁₀, and R₁₂, are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;

R₂, R₃, R₅ and R₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;

Ar is an aromatic moiety which when included in Formula (II) forms a multi-substituted aromatic hydrocarbon or a multi-substituted heteroaromatic group;

(r), (s), (t), and (u) are independently zero or one; and

(m) and (p) are independently zero a positive integer, preferably p is 1-6, and more preferably 1.

The benzyl elimination-based releasable linkers (RNL) employed in the compounds described herein are described in commonly-assigned U.S. Pat. Nos. 6,180,095, and 6,720,306, the contents of which are incorporated herein by reference.

In particular aspects of the compounds described herein, R₂ and R₆ are independently C₁₋₆ alkyls. Preferably, R₂ and R₆ are both methyl or methoxy. In other preferred aspects, R₃ and R₅ are hydrogen, hi another preferred aspect, R₁ and R₄ are independently selected from the group consisting of hydrogen, CH₃ and CH₂CH₃.

For purposes of the present invention, bifunctional linking moieties defined herein as L₁ can be selected from among:

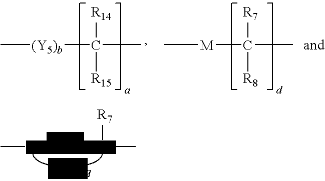

wherein:

M is X or Q
wherein
X is an electron withdrawing group;
Q is a moiety containing a free electron pair positioned three to six atoms from

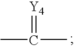

(a) and (d) are independently zero or a positive integer;

(b) is zero or one;

(q) is three or four;

R₇, R₈, R₁₄, and R₁₅ are independently selected from the group which defines R₉; and

Y₅ is O, S, or NR₁₂.

In some preferred aspects, R₂, R₃, R₅ and R₆, are not all H when (m) and (d) are both zero. In some particular embodiments, R₇ and R₈ include substituted C₁₋₆ alkyl selected from the group consisting of carboxyalkyls, aminoalkyls, dialkylaminos, hydroxyalkyls and mercaptoalkyls. In yet preferred embodiments, X is selected from the group consisting of O, NR₁₂, S, SO and SO₂, and preferably, O and NR₁₂ and Q is selected from the group consisting of C₂₋₄ alkyls, cycloalkyls, aryls, and aralkyl groups, Q is substituted with a member of the group consisting of NH, NR₁₂, O, S, CH₂CH(O)—N(H)—, and ortho-substituted phenyls. In further particular embodiments, (d) is an integer from 1 to about 12 and preferably 1 or 2.

In an alternative aspect, the releasable linkers employ an amino acid corresponding to

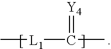

The amino acid residue can be among naturally occurring and non-naturally occurring amino acids, Derivatives and analogs of the naturally occurring amino acids, as well as various art-known non-naturally occurring amino acids (D or L), hydrophobic or non-hydrophobic, are also contemplated to be within the scope of the invention. A suitable non-limiting list of the non-naturally occurring amino acids residues includes 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, n-ethylglycine, N-ethylasparagine, 3-hydroxyproline, 4-hydroxyproline, isodesmosine, allo-isoleucine, N-methyl glycine, sarcosine, N-methyl-isoleucine, 6-N-methyl-lysine, N-methylvaline, norvaline, norleucine, and ornithine. Some preferred amino acid residues are selected from glycine, alanine, methionine or sarcosine, and more preferably, glycine.

L₁ is preferably

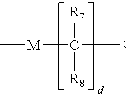

wherein all variables are as previously defined.

Alternatively, L₁ can be selected from among

—[C(═O)]_{v′}(CR₃₂R₃₃)_{w′}—,

—[C(═O)]_{v′}O(CR₃₂R₃₃)_{w′}O—,

—[C(═O)]_{v′}O(CR₃₂R₃₃)_{w′}NR₃₆—,

—[C(═O)]_{v′}O(CR₃₃R₃₃O)_{w′}NR₃₆—,

—[C(═O)]_{v′NR}₃₁(CR₃₂R₃₃)_{w′}—,

—[C(═O)]_{v′}NR₃₁(CR₃₂R₃₃)_{w′}O—,

—[C(═O)]_{v′}NR₃₁(CR₃₂R₃₃O)_{w′}—,

—[C(═O)]_{v′}NR₃₁(CR₃₂R₃₃O)_{w′}(CR₃₄R₃₅)_{y′}—,

—[C(═O)]_{v′}NR₃₁(CR₃₂R₃₃O)_{w′}(CR₃₄R₂₅)_{y′}O—,

—[C(═O)]_{v′}NR₃₁(CR₃₂R₃₃)_{w′}(CR₃₄CR₃₅O)_{y′}—,

—[C(═O)]_{v′}NR₃₁(CR₃₂R₃₃)_{w′}(CR₃₄CR₃₅O)_{y′}NR₃₆—,

—[C(═O)]ᵥ′NR₃₁(CR₃₂R₃₃)_{w′}NR₃₆—,

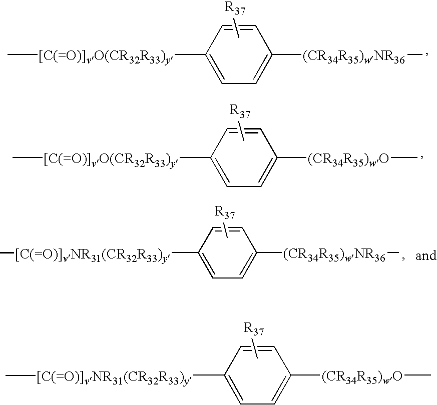

wherein:

R₃₁-R₃₇ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted-alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy,
wherein the substituents are selected from the group consisting of acyl, ammo, amido, amidine, araalkyl, aryl, azido, alkylmercapto, arylmercapto, carbonyl, carboxylate, cyano, ester, ether, formyl, halogen, heteroaryl, heterocycloalkyl, hydroxy, imino, nitro, thiocarbonyl, thioester, thioacetate, thioformate, alkoxy, phosphoryl, phosphonate, phosphinate, silyl, sulfhydryl, sulfate, sulfonate, sulfamoyl, sulfonamide, and sulfonyl;

(w′) and (y′) are independently selected from zero or positive integers, preferably 1 to 6; and

(v′) is 0 or 1.

The releasable linkers include an aromatic group, Ar which can be one of

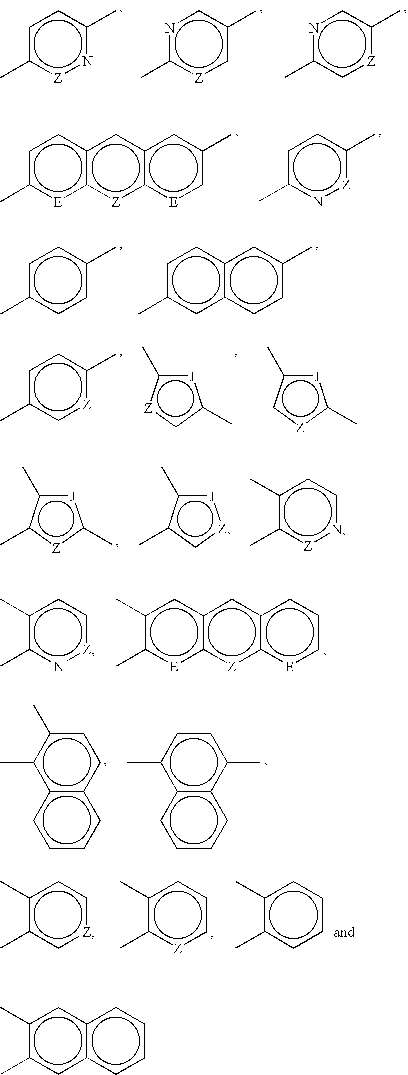

wherein

J is O, S, or NR₁₃;

E and Z are independently CR₁₃ or NR₁₃; and

R₁₃ is selected from the same group as mat which defines R₉.

It will be understood that the polymer conjugates of indenoisoquinoline can be prepared in alternative aspects of the invention with any of the RNL-type activated PEG linkers available from Enzon Pharmaceuticals, Inc. including those described in the aforementioned U.S. Pat. No. 6,180,095.

The present invention can employ alternative releasable linker systems such as tertiary methyl lock (TML)-based and bicine-based systems. Such alternative releasable linker systems including TML are described in U.S. Pat. Nos. 5,965,119, 6,624,142 and 6,303,569, the contents of which are incorporated herein by reference. Bicine-based releasable linker systems are described in commonly assigned U.S. Pat. Nos. 7,122,189 and 7,087,229 and U.S. patent application Ser. Nos. 10/557,522, 11/502,108, and 11/011,818. The disclosure of each such patents and patent applications is incorporated herein by reference.

### D. Preparation of IndQ Polymer Conjugates

In further aspects of the invention, there are provided methods of preparing releasably-linked IndQ polymer conjugates. Generally, PEG conjugates of IndQ were made through selective protection of IndQ followed by conjugation with the activated PEG. The activated RNL-NHS derivatives were made as previously reported using for example the techniques described in the aforementioned '095 patent. It will be understood by those of ordinary skill that these PEGylation conditions can be used to conjugate most other IndQ derivatives and other PEG linkers.

Methods of preparing various indenoisoquinoline compounds are described in US Patent Application Publication No. 2006/0025595, the content of which are incorporated by reference.

The present invention provides methods of preparing compounds of Formula (III). The methods include

(a) providing an activated polymer having the structure:

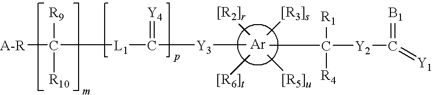

(b) reacting the activated polymer with a protected indenoisoquinoline having the structure:

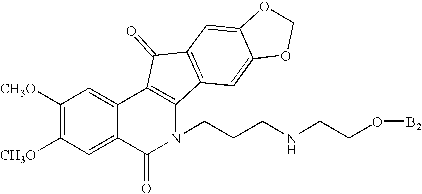

(c) removing the protecting group from the resulting intermediate of step (b) to form the compound of Formula (III):

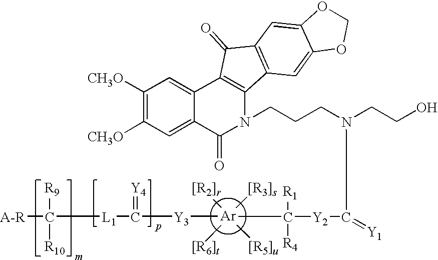

wherein

A is a capping group or

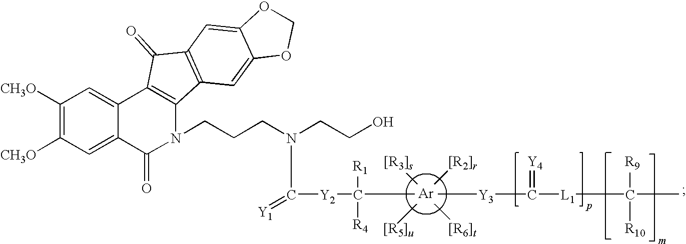

R is a substantially non-antigenic water-soluble polymer;

L₁ is a bifunctional linking moiety;

Y₁₋₄ are independently O, S, or NR₁₂;

R₁, R₄, R₉, R₁₀, and R₁₂, are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;

R₂, R₃, R₅ and R₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;

Ar is an aromatic moiety which when included in Formula (II) forms a multi-substituted aromatic hydrocarbon or a multi-substituted heteroaromatic group;

(r), (s), (t), and (u) are independently zero or one:

(m) and (p) are independently zero or a positive integer;

B₁ is a leaving group; and

B₂ is a protecting group.

Leaving groups or activating groups are known to those of ordinary skill and include, for example, p-nitrophenoxy, thiazolidinyl thione, N-hydroxysuccinimidyl or other suitable leaving or activating groups such as, N-hydroxybenzotriazolyl, halogen, N-hydroxyphthalimidyl, imidazolyl, O-acyl ureas, pentafluorophenol or 2,4,6-tri-chlorophenol or other suitable leaving groups apparent to those of ordinary skill. A non-limiting list of suitable OH-protecting groups to protect OH of IndQ is among TBDPSCl, TBDMSCl and TMSCl. Other suitable protecting groups well known to artisans in the art are with the scope of the invention.

Alternatively, the compounds described herein can be prepared by conjugating IndQ to a RNL linker, followed by PEGylation. It will be understood mat the polymer conjugates of indenoisoquinoline can be prepared in alternative aspects of the invention with any of the RNL-type activated PEG linkers available from Enzon Pharmaceuticals, Inc. including those described in U.S. Pat. No. 6,180,095.

As described in Scheme 1 shown below, various PEGs including 20kmPEG-RNL9-NHS, 20kΔPEG-RNL9-NHS, and 40kΔPEG-RNL9-NHS (the RNL9-NHS portion is shown below within the parenthesis), were attached selectively to the secondary amine group of MJ III 65 (NSC 706744), hereinafter “IndQ” by using a protecting group on OH. Following the deprotection, PEGylated IndQ conjugates have been successfully made. RNL9-NHS:

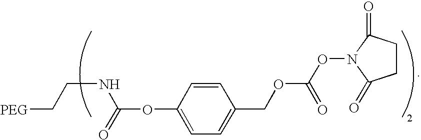

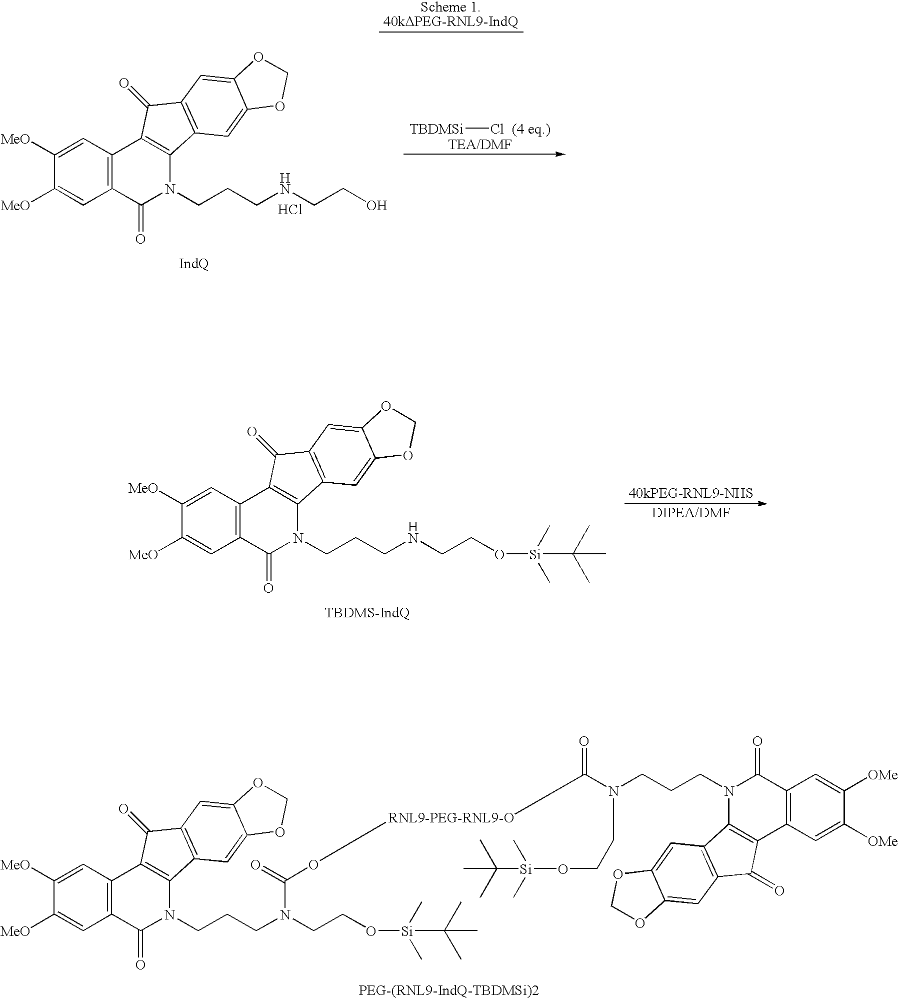

For purposes of the present invention, activating groups or leaving groups are to be understood as those groups which are capable of reacting with a secondary amine group found on IndQ.

In some aspects of the invention, the activated polymers including releasable linker systems are among:

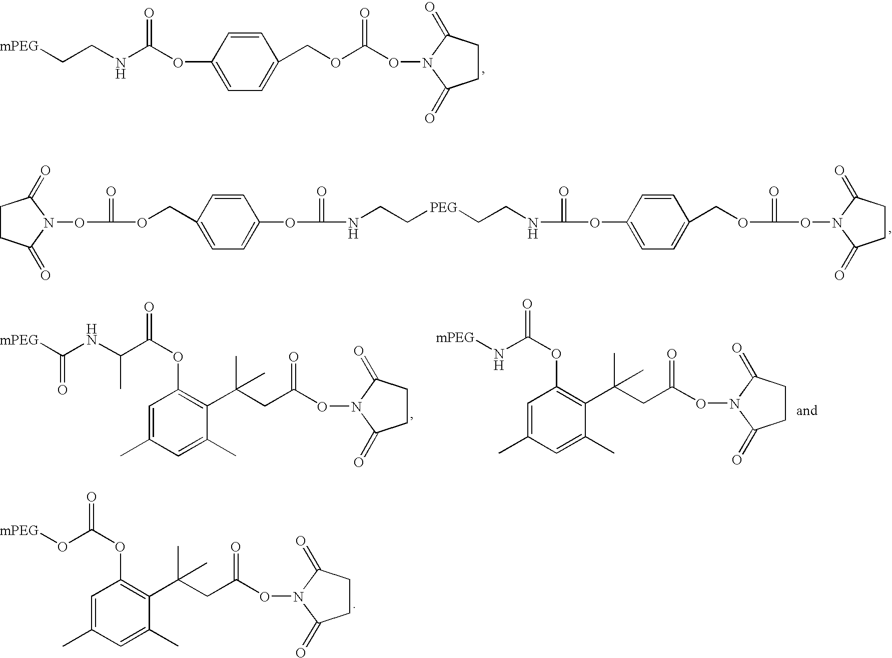

In one alternative aspect of the invention, the IndQ polymer conjugates include certain bicine-based releasable linker systems such as those described in commonly assigned U.S. Pat. Nos. 7,122,189 and 7,087,229 and U.S. patent application Ser. Nos. 10/557,522, 11/502,108, and 11/011,818. A few of such activated polymers include:

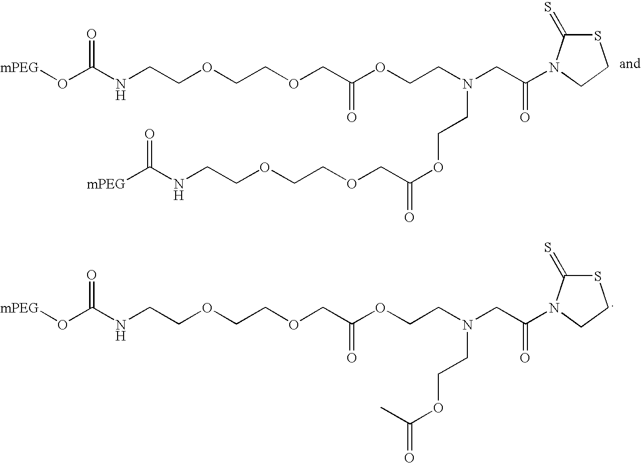

### E. Preferred Compounds

Some particular embodiments of the compounds described herein are selected from among

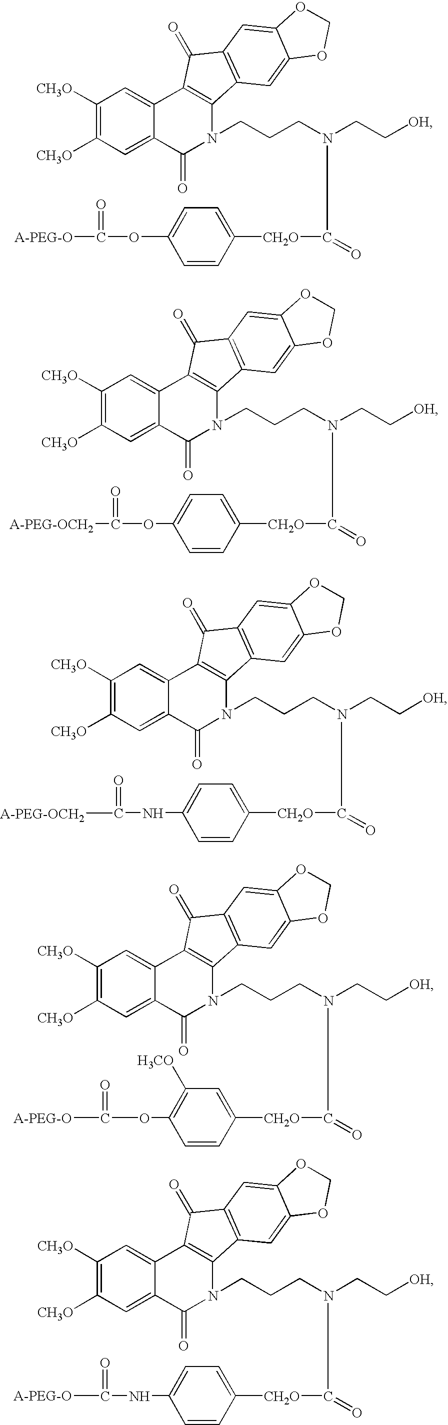

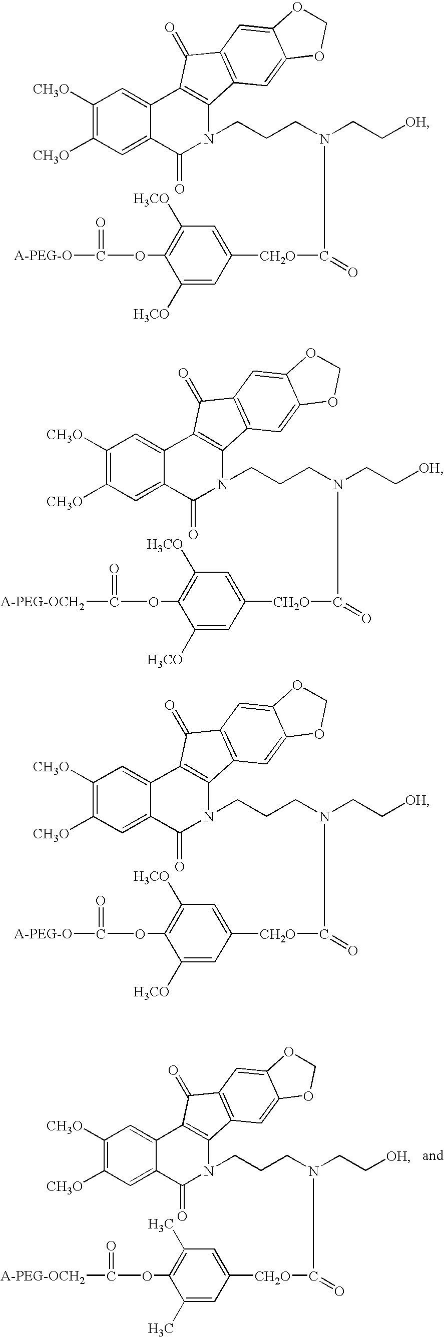

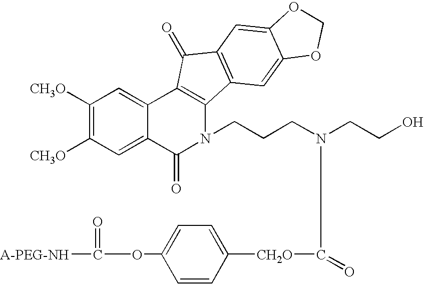

wherein PEG has the formula:

—(CH₂CH₂O)ₓCH₂CH₂— and
(x) is an integer from about 10 to about 2,300.
   Preferably, the compounds of the invention include

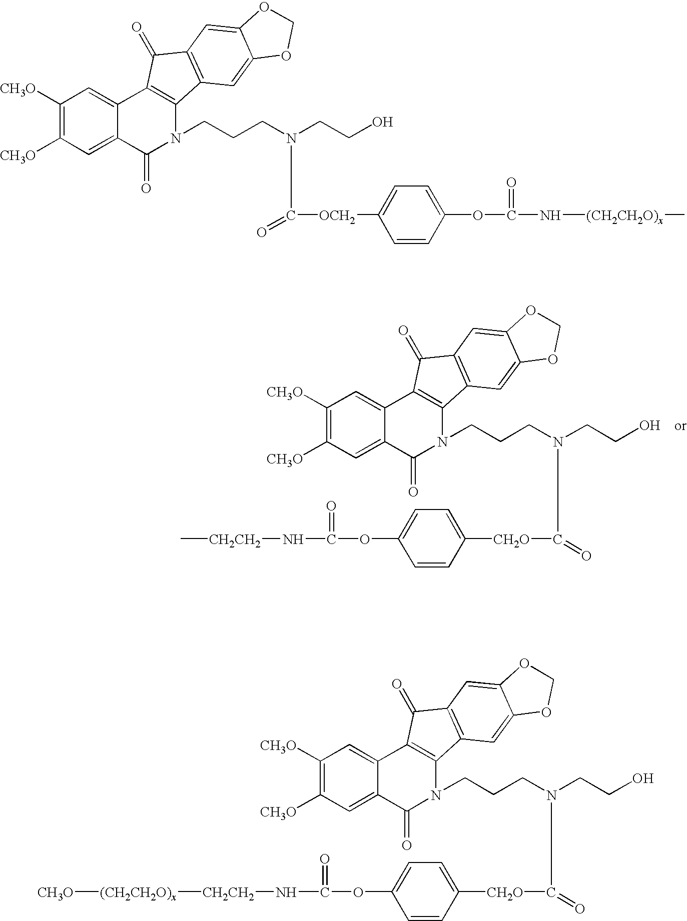

wherein (x) is an integer from about 10 to about 2300.

In yet another aspect of the present invention, there are provided pharmaceutically acceptable salts of compounds of Formula (III). In still further aspects of the invention, there are provided pharmaceutically-acceptable formulations containing an effective amount of compounds of Formula (IIII) or salt thereof.

### F. Methods of Treatment

In yet another aspect, the present invention provides methods of treating cancers. The methods Include administering an effective amount of a compound of Formula (III) to a patient in need thereof. The conditions which can be treated, include cancer or tumor or generally a condition calling for administration of a topoisomerase I inhibitor and/or an indenoisoquinoline.

In one particular aspect, the methods of treatment include administering compounds having the structure:

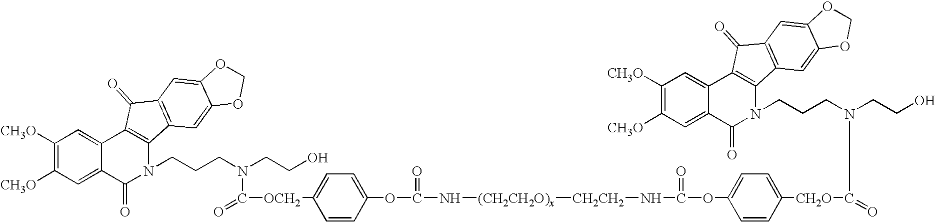

wherein (x) is an integer from about 10 to about 2300. Preferably, (x) is an integer selected so that the PEG has molecular weight of from about 20,000 to about 60,000 daltons, and more preferably about 40,000 daltons.

Determination of a therapeutically effective amount is well within the capability of those skilled in the art, especially in light of the disclosure herein.

For any compound used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients. The amount of the composition, e.g., used as a prodrug, that is administered will depend upon the parent molecule included therein. Generally, the amount of prodrug used in the treatment methods is that amount which effectively achieves the desired therapeutic result in mammals. Naturally, the dosages of the various prodrug compounds will vary somewhat depending upon the parent compound, rate of in vivo hydrolysis, molecular weight of the polymer, etc. In addition, the dosage, of course, can vary depending upon the dosage form and route of administration, hi general, however, the compounds described herein can be administered in amounts ranging from about 1 to about 100 mg/kg/week and preferably from about 2 to about 60 mg/kg/week. The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. Additionally, toxicity and therapeutic efficacy of the compounds described herein can be determined by standard pharmaceutical procedures in cell cultures or experimental animals using methods well-known in the art. In one particular aspect, the invention provides that methods of treating cancers or topoisomerase I inhibitor-related diseases includes administering the compounds described herein in amounts of from about 5 mg/kg/dose to about 20 mg/kg/dose equivalent to IndQ.

In one particular aspect, the treatment of the present invention includes administering the compounds described herein in an amount of from about 5 to about 20 mg/kg/dose or from about 10 to about 30 mg/kg/dose to a mammal having cancers or topoisomerase I inhibitor-related diseases.

The compositions may be administered once daily or divided into multiple doses which can be given as part of a multi-week treatment protocol. The precise dose will depend on the stage and severity of the condition, the susceptibility of the tumor to the polymer-prodrug composition, and the individual characteristics of the patient being treated, as will be appreciated by one of ordinary skill in the art.

In all aspects of the invention where polymeric conjugates are administered, the dosage amount mentioned is based on the amount of IndQ rather than the amount of polymeric conjugate administered. It is contemplated that the treatment will be given for one or more days until the desired clinical result is obtained. The exact amount, frequency and period of administration of the compound of the present invention will vary, of course, depending upon the sex, age and medical condition of the patent as well as the severity of the disease as determined by the attending clinician.

Still further aspects include combining the compound of the present invention described herein with other anticancer therapies for synergistic or additive benefit.

### G. Compositions/Formulations

Pharmaceutical compositions containing the polymer conjugates described herein may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable earners comprising excipients and auxiliaries which facilitate processing of the active compounds into preparations which can be used pharmaceutically. Proper formulation is dependent upon the route of administration chosen.

For injection, including, without limitation, intravenous, intramuscular and subcutaneous injection, the compounds of the invention may be formulated in aqueous solutions, preferably in physiologically compatible buffers such as physiological saline buffer or polar solvents including, without limitation, a pyrrolidone or dimethylsulfoxide.

The compounds may also be formulated for parenteral administration, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of die compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the particular compound, additional stabilization strategies may be employed.

### EXAMPLES

The following examples serve to provide further appreciation of the invention but are not meant in any way to restrict the effective scope of the invention. The underlined and bold-faced numbers recited in the Examples correspond to those shown in scheme 2. Abbreviations are used throughout the examples such as, ACN (acetonitrile), DCM (dichloromethane), DMF (N,N′-dimethylformamide), DSC (disuccinimidyl carbonate), IPA (isopropanol), and TEA (triethylamine).

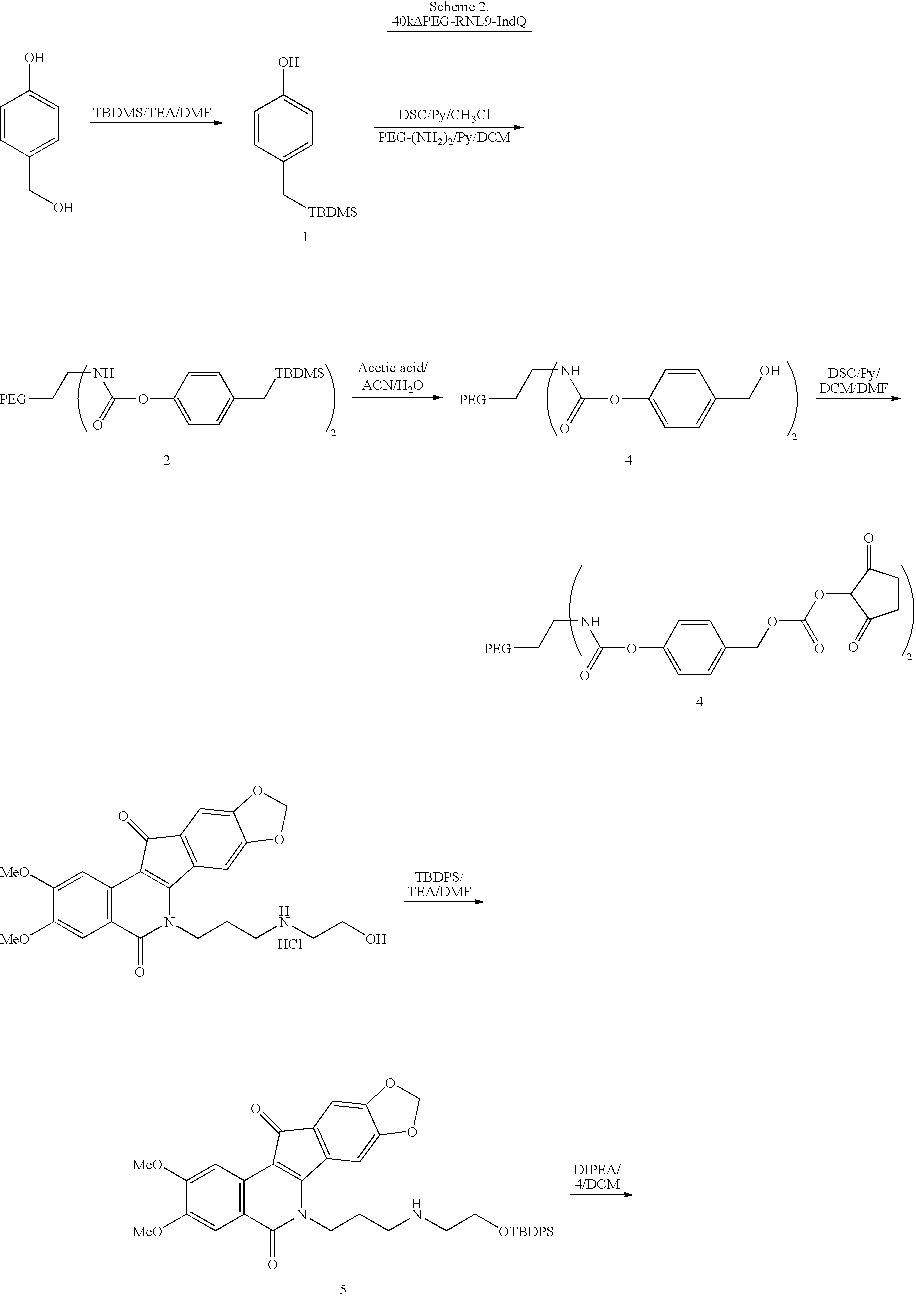

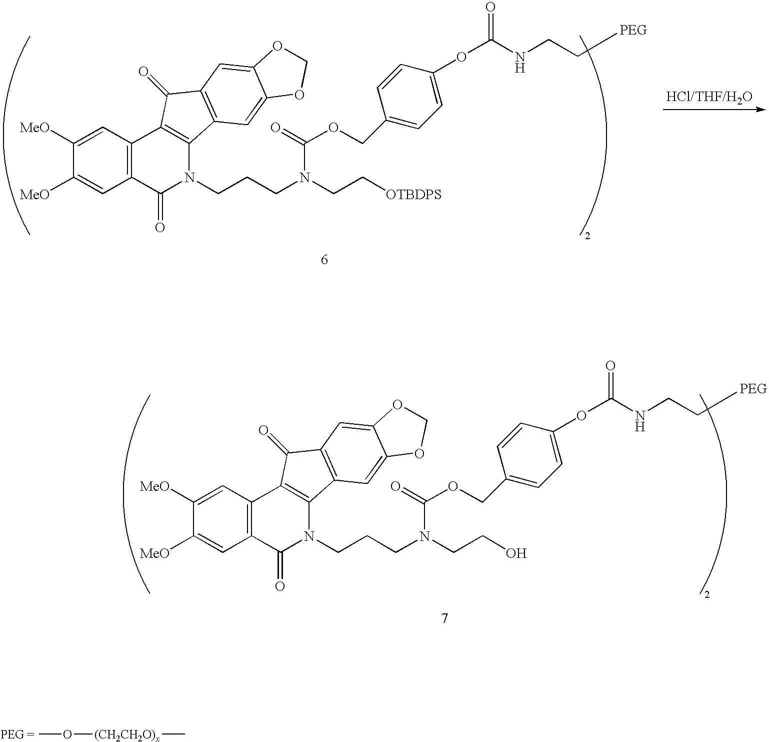

### Example 1

### General

All reactions were conducted under an atmosphere of dry nitrogen. Commercial regents and anhydrous solvents were used without further purification. Indenoisoquinoline compound (IndQ, MJ III 65 or NSC 706744) was supplied by National Cancer Institute and Purdue University. NMR spectra were recorded at a Varian Mercury 300 MHz NMR spectrometer using deuterated solvent indicated. Chemical shifts (d) are reported in parts per million (ppm) downfield from tetramethylsilane (TMS) and coupling constants (J values) are given in hertz (Hz). Reaction progress and in vitro hydrolysis of PEGylated Indenoisoquinoline in rat plasma were monitored by a Waters 2420 HPLC with a UV detector monitored at 275 nm, on a Phenomenex Jupiter 300A 250×4.6 mm C18 column using a linear gradient of acetonitrile in water with 0.05% TFA. Mass spectra were obtained from an Agilent Mass Spectrometer.

### Example 2

### Compound 1

A solution of 4-hydroxylbenzyl alcohol (10.0 g, 80.6 mmol) and tert-butyldimethylsilyl chloride (13.4 g, 88.9 mmol) in DMF (50 mL) was cooled to 0° C. in an ice bath, followed by addition of a solution of TEA (44.6 mmol) in DMF (100 mL). The reaction mixture was allowed to warm up to room temperature and stirred over night. After the reaction completion was con filmed by TLC, the reaction mixture was concentrated by evaporation under reduced pressure, followed by extraction with DCM (200 mL) and 10% NaHCO₃ (150 mL). The organic layer was dried over anhydrous MgSO₄, and concentrated by rotary evaporation. This product was further purified by chromatography on silica gel to give compound 1 (9.5 g): ¹H NMR (300 MHz, CDCl₃) d 7.08 (d, 2H, J= 8.3), 6.67 (d, 2H, J= 8.4), 4.99 (s, 1H), 4.57 (s, 1H), 0.93 (s, 9H), 0.091 (s, 6H); ¹³C NMR (75.4 MHz, CDCl₃) d 154.4, 133.4, 127.8, 115.0, 64.8, 26.0, 18.5, −5.1.

### Example 3

### Compound 2

Disuccinimidyl carbonate (DSC, 0.70 g, 2.73 mmol) was suspended in chloroform (50 mL) containing compound 1 (0.71 g, 3.0 mmol) and pyridine (0.26 mL, 3.25 mmol). The mixture was refluxed overnight, then cooled to room temperature, followed by addition of a solution of 40kPEG-(NH₂)₂ (10 g, 0.25 mmol) and pyridine (0.26 mL, 3.25 mmol) in DMF (30 mL). The reaction mixture was stirred at room temperature overnight, and the mixture was concentrated by evaporation under reduced pressure. The crude product was precipitated by ether, subjected to crystallization from 20% DMF/IPA to give compound 2 (9.5 g): ¹³C NMR (75.4 MHz, CDCl₃) d 154.5, 149.6, 138.0, 126.6, 121.1, 64.3, 40.9, 25.9, 18.3, −5.2.

### Example 4

### Compound 3

Compound 2 (8.5 g) was stirred at room temperature overnight in a mixture of ACN (40 mL), H₂O (20 mL) and acetic acid (100 mL), followed by extraction with DCM (100 mL). The organic phase was concentrated by rotary evaporation, precipitated with ether, and washed with ether to yield compound 3 (8.0 g): ¹³C NMR (75.4 MHz, CDCl₃) d 154.4, 150.0, 138.1, 127.5, 121.3, 64.2, 40.9.

### Example 5

### ^{40k}PEG-(RNL9-NHS)₂ (Compound 4)

DSC (0.77, 3.0 mmol) was suspended in a solution of DCM (100 mL) and DMF (10 mL) containing compound 3 (7.5 g, 0.188 mmol). The mixture was cooled to 0° C.˜−10° C., followed by addition of pyridine (0.25 mL, 3.0 mmol). The mixture was allowed to warm to room temperature overnight, and the mixture was concentrated by evaporation under reduced pressure. The crude product was precipitated with ether, and recrystallized from 20% (v/v) DMF/IPA to produce compound 4 (6.5 g): ¹³C NMR (75.4 MHz, CDCl₃) d 168.3, 154.1, 151.5, 151.2, 129.8, 129.6, 121.7, 40.9, 25.4.

### Example 6

### IndQ-TBDPS (Compound 5)

To a suspension of IndQ (1.0 g, 2.21 mmol) and tert-butyldiphenylsilyl chloride (2.82 g, 10.3 mmol) in 100 mL of anhydrous DMF was added a mixture of 2.32 mL (16.7 mmol) of TEA and 60 mL of DMF at room temperature. The reaction mixture was vigorously stirred at 50° C. overnight. When IndQ was disappeared, the mixture was concentrated by evaporation under reduced pressure. The residue was loaded onto an open silica gel column using a mixture of ethyl acetate, DCM and methanol as elute to yield compound 5 (0.55 g) with purity of 94.7%: ¹H NMR (300 MHz, DMSO-d₆) d 7.84 (s, 1H), 7.63 (m, 4H) 7.61 (s, 1H), 7.44 (m, 7H), 6.08 (s, 2H), 4.84 (m, 2H), 3.89 (s, 3H), 3.83 (s, 3H), 3.73 (t, 2H), 2.72 (m, 2H), 2.66 (m, 2H), 1.82 (m, 2H), 0.99 (s, 9H) ppm; ESI-MS, 691.36 [M+1]⁺.

### Example 7

### ^{40k}TEG-(RNL9-IndQ-TBDPS)₂ (Compound 6)

A solution of compound 4 (6.0 g, 0.15 mmol), compound 5 (0.45 g, 0.65 mmol) and DIPEA (0.5 ml, 2.87 mmol) was stirred at room temperature overnight, followed by removal of solvents by evaporation under reduced pressure. The residues were precipitated with ether. The precipitate was collected by vacuum filtration and washed with ether. This crude product was recrystallized from 20% DMF/IPA twice at 65° C. to give compound 6 (5.4 g).

### Example 8

### ^{40k}PEG-(RNL9-IndQ)₂ (Compound 7)

Compound 6 (5.4 g) was dissolved in 2.5 N HCl in 50% aqueous THF solution (200 mL). The solution was stirred at room temperature. After the disappearance of compound 6 was confirmed by HPLC, the reaction mixture was evaporated to remove THF, followed by extraction with DCM. The organic phase was concentrated, followed by precipitation with ether to give crude product. The produce was subjected to crystallization from 20% DMF/IPA at 65° C. to produce compound 7 (4.4 g) with purity of 99%: ¹³C NMR (75.4 MHz, CDCl₃) d 189.1, 162.0, 156.4, 154.6, 152.9, 151.2, 150.6, 148.8, 148.6, 133.0, 132.1, 130.0, 128.8, 127.9, 121.4, 116.6, 107.7, 105.1, 102.7, 102.5, 66.7, 61.0, 56.1, 55.9, 50.8, 46.1, 42.5, 40.9, 28.6.

### Example 9

### Hydrolysis of IndQ-RNL9-PEG Conjugates in Rat Plasma

To each vial (1.5 mL) was added 0.1 mL of solution of PEGylated IndQ conjugate with concentration of 10 mg/mL in methanol. After methanol was removed under reduced pressure, 0.1 mL of rat plasma was added to each vial to initiate the hydrolysis. The each vial was vortexed for 0.5 min and placed immediately into an incubator of 37° C. At the time intervals of 0, 0.5, 2, 4, 6, and 24 h, to the selected vials was added 0.4 mL of a mixture of MeOH and ACN (1:1) to quench the hydrolysis. The quenched mixture was filtrated through 0.45 micron filter membrane, and 10 μL of the filtrate was injected into HPLC system. The results are shown below in Table 1.

### Example 10

### Determination of IndQ %

Active IndQ % by weight was determined for fire compounds described herein. For example, the amount of IndQ in 40kΔPEG-IndQ including RNL9 was measured on Bio UV-Visible spectrometer using IndQ with purity of 99.5% as external standards. The IndQ (10 mg) was dissolved in 25 mL of 90% DMF, followed by sonication for 30 min to make the IndQ stock solution at a concentration of 0.885 mmol/mL. The IndQ standard solutions used for standard curve measurement was made by diluting the stock IndQ solution to concentrations from 0.05 μmol/mL to 0.030 μmol/mL. Each of PEG-RNL9-IndQ compounds tested (10 mg) was dissolved in 1 mL of 90% aqueous DMF. The absorbance of IndQ and PEGylated IndQ solutions was measured at 290 nm to calculate the amount of IndQ. See Table 1.

TABLE 1
Pharmacokinetic Profiles of PEG-RNL9-IndQ Conjugates

| | 20 kmPEG- | 20 kΔPEG- | | 40 kΔPEG- | |
|---|---|---|---|---|---|
| Compd. | IndQ | IndQ | | IndQ | |
| UV# | 0.74 | 1.77 | | 2.44 | |
| % of Active IndQ | 1.84 | 4.4 | | 3.0 | |
| by weight | | | | | |
| Purity by GPC | — a | 28,072 | (88%) | 45,164 | (90%) |
| Purity by HPLC | — a | 95% | | 90% | |
| Solubility in saline | 162 | 53 | (2.3*) | 33 | (1.0*) |
| (mg/mL) | | | | | |

| | | | |
|---|---|---|---|
| Stability in saline at | — a | <1% | — a |
| RT for 4 h (amount | | | |
| of decomposition) | | | |
| t_{1/2} in rat plasma at | — a | 4.2 h | 4.3 h |
| 37° C. (h) | | | |

a not available
*equivalent to IndQ
Formulation in saline at pH = 7.0

The table shows that the compounds described herein allow IndQ to be solubilized and stable in saline solution. PEGylation of indenoisoquinoline compound using the customized releasable PEG linkers has successfully solubilized the very insoluble IndQ (NSC 706744). For example, less than 1% of 20kΔPEGylated IndQ including RNL9 in saline solution was decomposed at room temperature for 4 hours. PEGylated IndQ including RNL9 shows about 4.2 hours half-life in rat plasma in vitro.

### Example 11

### Hollow Fiber Assay

A standard panel of 12 tumor cell lines is used for routine hollow fiber screening. These include NCI-H23, NCI-H522, MDA-MB-231, MDA-MB-435, SW-620, COLO 205, LOX, UACC-62, OVCAR-3, OVCAR-5, U251 and SF-295. A total of 3 different tumor lines are prepared for each experiment so that each mouse receives 3 intraperitoneal implants (1 of each tumor line) and 3 subcutaneous implants (1 of each tumor line). Each compound tested is administered by intraperitoneal injection at 2 dose levels. The percent net growth for each cell line in each treatment group is calculated and compared to the percent net growth in the vehicle treated controls. A 50% or greater reduction in percent net growth in the treated samples compared to the vehicle control samples is considered a positive result. Each positive result is given a score of 2 and all of the scores are totaled for a given compound tested. The maximum possible score for a compound tested is 96 (12 cell lines×2 sites×2 dose levels×2 [score]). A compound is considered for xenograft testing if it has a combined ip+sc score of 20 or greater, a sc score of 8 or greater, or produces cell death of any cell line at either dose level evaluated.

The in vivo hollow fiber assay (HFA) in mice was conducted per previously published methods using the 20 k ΔPEG-RNL9-IndQ at equivalent IndQ active doses of 12 and 18 mg/kg/dose and 40 k ΔPEG-RNL9-IndQ at equivalent IndQ active doses of 9 and 12 mg/kg/dose. For comparison, NSC 706744 (native or unmodified) was evaluated at doses of 100 and 150 mg/kg/dose which are the highest doses routinely tested.

The amounts of the compounds administered are based on formulations such as

those providing, for example, a dose of about 16 mg/kg. Therefore:

Calculation: ^{20k}ΔPEG-RNL9-IndQ

Dose of 16 mg/kg

Using a mouse, for example, weight: 25 g

0.4 mg of IndQ per mouse
(16 mg/1000 g)*25 g=0.4 mg

10.8 mg of IndQ-PEG conjugate per mouse
% of IndQ by weight: 3.7%
0.4 mg/3.7%= 10.8 mg

0.20 ml of IndQ-PEG with a concentration of 53 mg/mL per mouse
Solubility= C=53 mg/ml
V=10.8/53= 0.20 mL

Similarly:

Calculation: ^{40k} ΔPEG-RNL9-IndQ

Dose of 16 mg/kg

Using a mouse weight: 25 g

0.4 mg of IndQ per mouse
(16 mg/1000 g)*25 g=0.4 mg

18.2 mg of IndQ-PEG conjugate per mouse
% of IndQ by weight: 2.2%
0.4 mg/2.2%= 18.2 mg

0.55 ml of IndQ-PEG with a concentration of 32.9 mg/mL per mouse
Solubility= C=32.9 mg/ml
V= 18.2/32.9=0.55 mL
   The results of the in vivo HFA activity are set forth in Table 2.

TABLE 2
In Vivo HFA Activity

| | | | | Cell | |
|---|---|---|---|---|---|
| | IP | SC | IP + SC | Death | Activity |
| ^{20k}ΔPEG-RNL9-IndQ | 12 | 4 | 16 | Yes | Active |
| ^{40k}ΔPEG-RNL9-IndQ | 28 | 10 | 38 | Yes | Active |
| Temozolomide* | 20 | 8 | 28 | Yes | Active |
| Taxol* | 36 | 6 | 42 | Yes | Active |

*Historic data for comparison

The in vivo studies showed that both PEGylated IndQ compounds had excellent activity in contrast to comparable doses of the parent, NSC 706744. Using the published scoring comparison, NSC 706744 scored 6/48 points in IP fibers and 6/48 points in SC fibers at doses of 100 and 150 mg/kg which are significantly higher (based upon available active agent) than the ^{20k} ΔPEG-RNL9-IndQ doses and ^{40k} ΔPEG-RNL9-IndQ. ^{20k} ΔPEG-RNL9-IndQ tested scored of 12/48 IP and 4/48 SC. The doses of ^{40k}ΔPEG-RNL9-IndQ tested resulted in significantly higher scores of 28/48 IP and 10/48 SC in contrast to the much higher doses of NSC 706744. The 40 k PEG total score of 38/96 places it in the top 3% of the 3604 compounds evaluated in the hollow fiber assay to date. The in vivo HFA study showed that enhanced anti-tumor efficacy can be achieved through customized PEGylation.

We claim:

1. A compound of Formula (I) 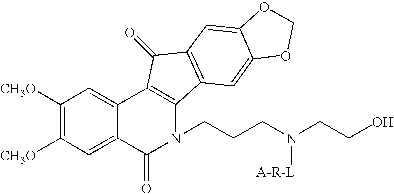
wherein
A is a capping group 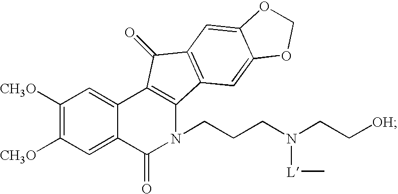
R is a substantially non-antigenic water-soluble polymer; and
L and L′ are independently selected releasable linkers.

2. The compound of claim 1, wherein the independently selected releasable linkers are of the Formula (II) 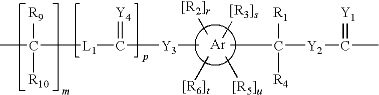
wherein
L₁ is a bifunctional linking moiety;
Y₁₋₄ are independently O, S, or NR₁₂;
R₁, R₄, R₉, R₁₀, and R₁₂, are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;
R₂, R₃, R₅ and R₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;
Ar is an aromatic moiety which when included in Formula (II) forms a multi-substituted aromatic hydrocarbon or a multi-substituted heteroaromatic group;
(r), (s), (t), and (u) are independently zero or one; and
(m) and (p) are independently zero a positive integer.

3. A compound of claim 1 having the Formula (III) 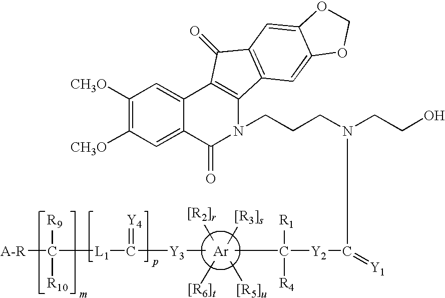
wherein
A is a capping group or 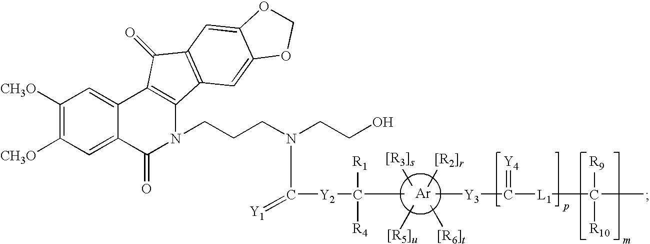
R is a substantially non-antigenic water-soluble polymer;
L₁ is a bifunctional linking moiety;
Y₁₋₄ are independently O, S, or NR₁₂;
R₁, R₄, R₉, R₁₀, and R₁₂, are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;
R₂, R₃, R₅ and R₆, are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;
Ar is an aromatic moiety which when included in Formula (II) forms a multi-substituted aromatic hydrocarbon or a multi-substituted heteroaromatic group;
(r), (s), (t), and (u) are independently zero or one; and
(m) and (p) are independently zero a positive integer.

4. The compound of claim 3, wherein the capping group is selected from the group consisting of hydrogen, NH₂, OH, CO₂H, C₁₋₆ alkyl, C₁₋₆ alkoxy, and dialkyl acyl urea alkyls.

5. The compound of claim 3, wherein the capping group is CH₃ or OCH₃.

6. The compound of claim 3, wherein A is

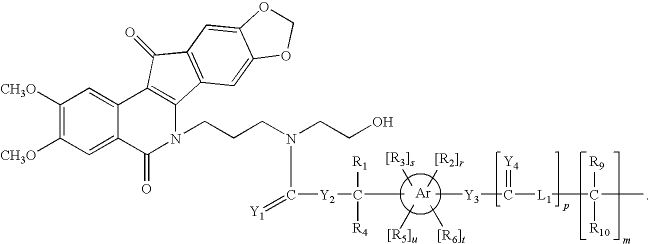

7. A compound of claim 3 having the formula

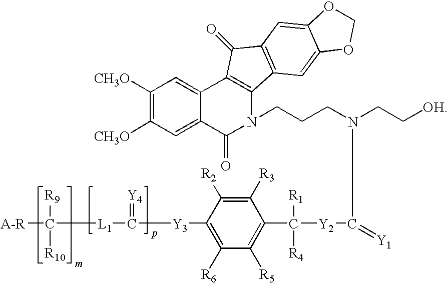

8. A compound of claim 7 having the formula

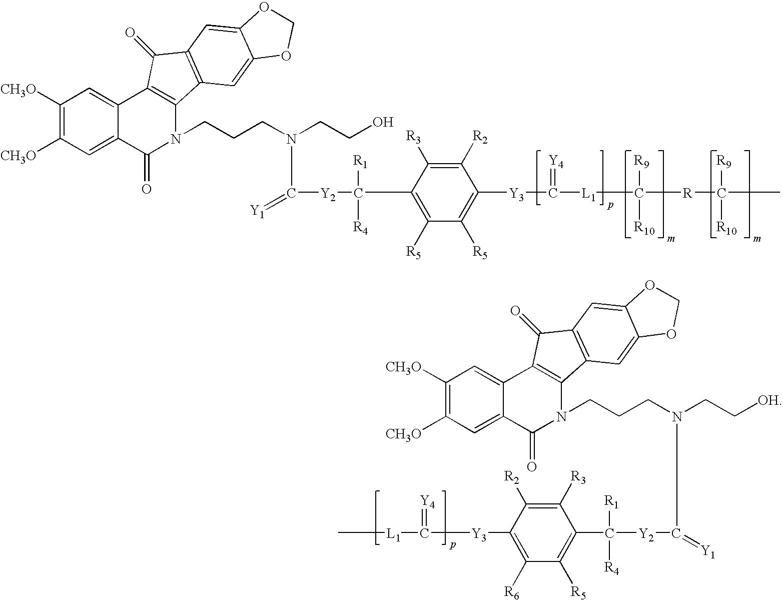

9. A compound of claim 3, having the formula

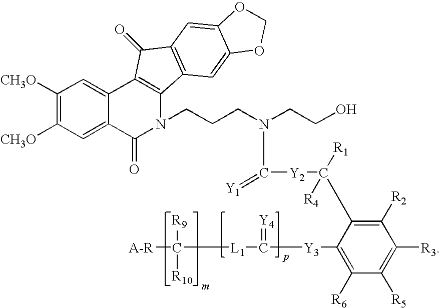

10. The compound of claim 3, wherein R comprises a linear, terminally branched or multi-aimed polyalkylene oxide.

11. The compound of claim 3, wherein R is selected from the group consisting of —C(═Y₂₁)—(CH₂)ₙ—O—(CH₂CH₂O)ₓ-A, and —C(═Y₂₁)—Y₂₂—(CH₂)ₙ—O—(CH₂CH₂O)ₓ-A
wherein
(n) is zero or a positive integer;
Y₂₁₋₂₂ are independently O, S or NR₁₂; and
(x) represents the degree of polymerization.

12. The compound of claim 10, wherein said polyalkylene oxide is a polyethylene glycol of the formula: —O—(CH₂CH₂O)ₓ—
wherein x is an integer from about 10 to about 2,300.

13. The compound of claim 10, wherein the polyalkylene oxide has an average molecular weight from about 2,000 to about 100,000 Daltons.

14. The compound of claim 10, wherein the polyalkylene oxide residue has an average molecular weight of from about 10,000 to about 80,000 daltons.

15. The compound of claim 10, wherein the polyalkylene oxide has an average molecular weight from about 20,000 to about 60,000 Daltons.

16. The compound of claim 10, wherein the polyalkylene oxide has an average molecular weight of about 40,000 Daltons.

17. The compound of claim 3, wherein (m) and (p) are zero.

18. The compound of claim 3, wherein R₁, R₄, R₉ and R₁₀ are all hydrogen.

19. The compound of claim 3, wherein Y₁₋₄ are O.

20. A compound of claim 3, selected from the group consisting of: 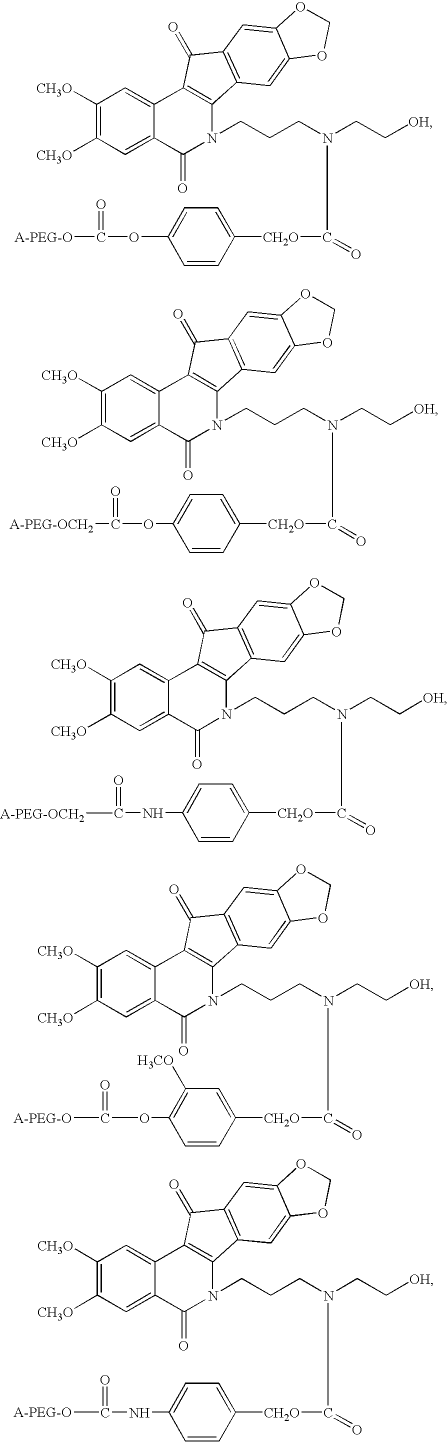 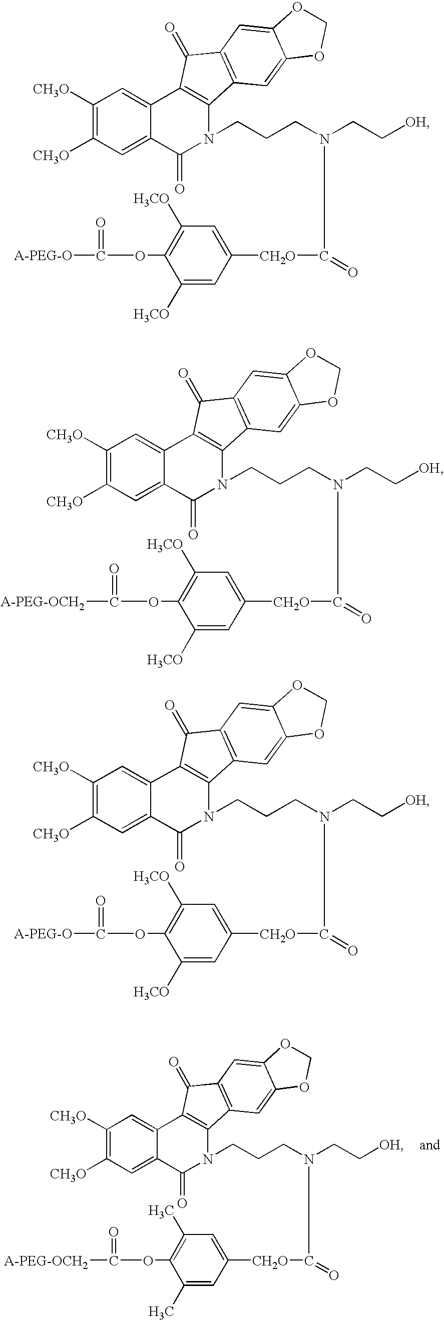 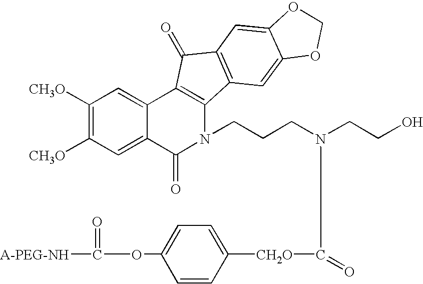
wherein PEG has the formula:
—(CH₂CH₂O)ₓ—CH₂CH₂—
wherein (x) is an integer from about 10 to about 2,300.

21. A compound of claim 3 is 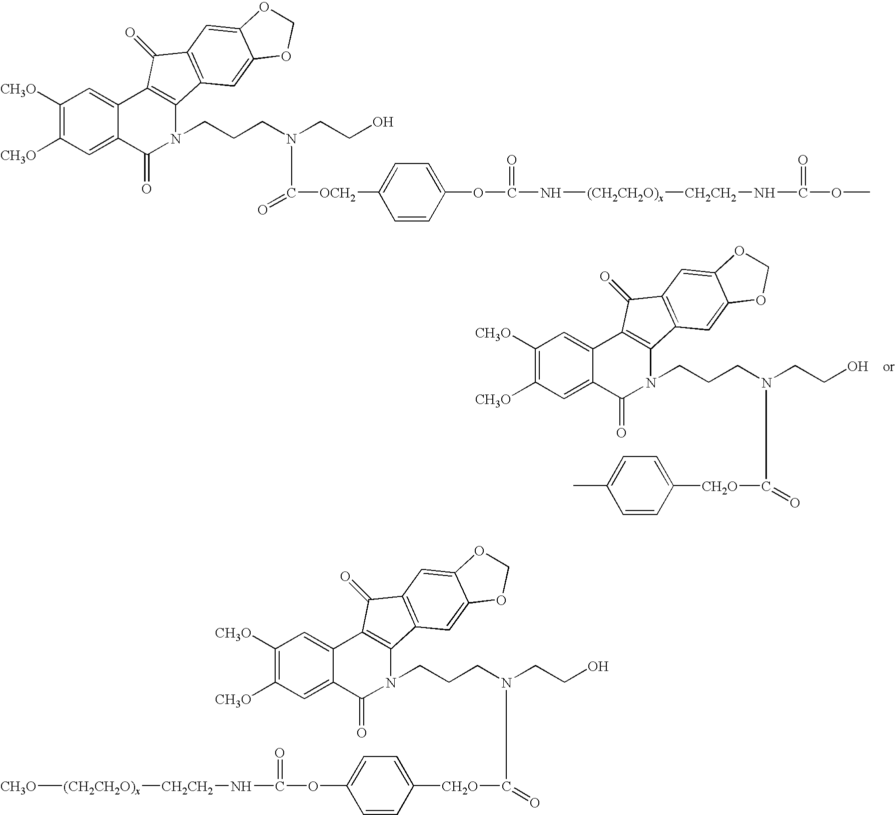
wherein x Is an integer from about 10 to about 2300.

22. A pharmaceutically acceptable formulation containing an effective amount of a compound of claim 3 or salt thereof.

23. A method of treating cancers, comprising administering an effective amount of a compound of claim 3 to a patient in need thereof.

24. The method of claim 23, wherein the compound of claim 3 is 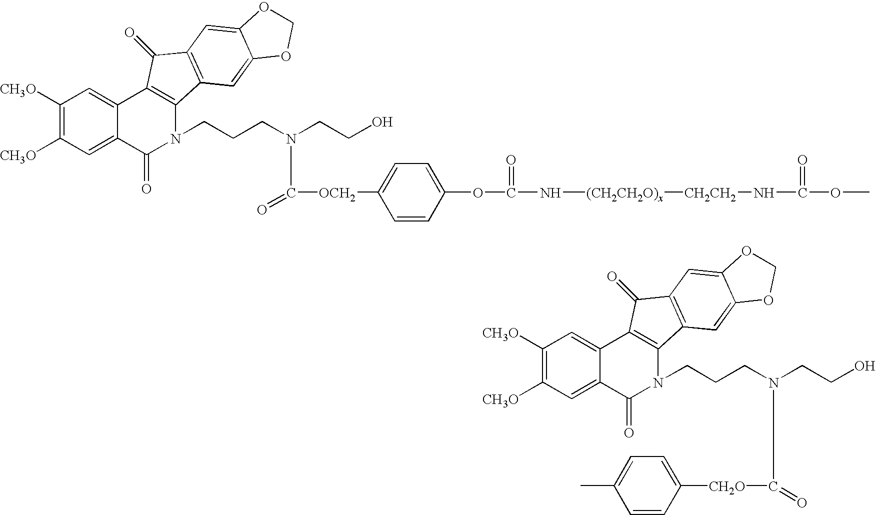
wherein x is an integer from about 10 to about 2300.

25. A method of preparing a compound of Formula (III), comprising;
(a) providing an activated polymer having the structure: 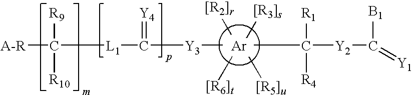
(b) reacting the activated polymer with a protected indenoisoquinoline having the structure: 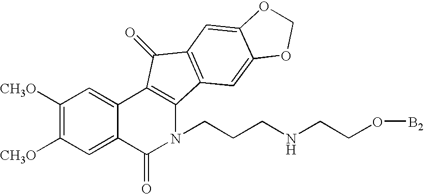
(c) removing the protecting group from the resulting intermediate of step (b) to form the compound of Formula (III): 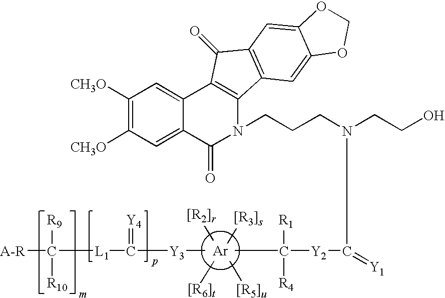
wherein
A is a capping group or 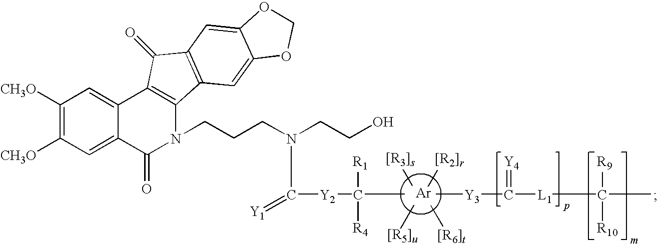
R is a substantially non-antigenic water-soluble polymer;
L₁ is a bifunctional linking moiety;
Y₁₋₄ are independently O, S, or NR₁₂;
R₁, R₄, R₉, R₁₀, and R₁₂, are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, and substituted C₁₋₆ heteroalkyls;
R₂, R₃, R₅ and R₆ are independently selected from the group consisting of hydrogen, C₁₋₆ alkyls, C₁₋₆ alkoxy, phenoxy, C₁₋₈ heteroalkyls, C₁₋₈ heteroalkoxy, substituted C₁₋₆ alkyls, C₃₋₈ cycloalkyls, C₃₋₈ substituted cycloalkyls, aryls, substituted aryls, aralkyls, halo-, nitro-, cyano-, carboxy-, C₁₋₆ carboxyalkyls and C₁₋₆ alkyl carbonyls;
Ar is an aromatic moiety which when included in Formula (II) forms a multi-substituted aromatic hydrocarbon or a multi-substituted heteroaromatic group;
(r), (s), (t), and (u) are independently zero or one;
(m) and (p) are independently zero or a positive integer;
B₁ is a leaving group; and
B₂ is a protecting group.
